(19) United States
(12) Patent Application Publication
(10) Pub. No.: US 2007/0154982 A1
(43) Pub. Date: Jul. 5, 2007

(54) Mammalian cell lines modified for the production of recombinant glycoproteins

(75) Inventors: Giancarlo Tonon, Milan (IT); Sabrina Pozzuolo, Sesto San Giovanni (IT)

Correspondence Address: DARBY & DARBY P.C., P. O. BOX 5257, NEW YORK, NY, 10150-5257, US

(73) Assignee: Keryos SPA, Gessate (IT)

(21) Appl. No.: 10/575,051

(22) PCT Filed: Sep. 27, 2004
(86) PCT No.: PCT/IT04/00529

§ 371 Date: Apr. 3, 2006

(30) Foreign Application Priority Data
Oct. 3, 2003 (IT) MI2003A001909

(51) Int. Cl. C12P 21/06; C12N 5/06; C07K 14/47

(52) U.S. Cl. 435/69.1; 435/358; 530/395

(57) ABSTRACT

Novel, genetically modified mammalian cell lines are described, which can produce glycoconjugates the oligosaccharide portions of which are more similar to the human one than those of the same glycoconjugates produced by cells which have not been genetically modified. The lines in question can be used for the production of recombinant glycoconjugates which are of therapeutic interest, in particular for the production of glycoproteins for use in human therapy, since the glycoconjugates produced in these modified lines have a lower immunogenic potential for man than corresponding glycoconjugates produced in cells which have not been genetically modified.

The present invention relates to novel, genetically modified mammalian cell lines. The modifications relate to genetic engineering interventions on the cell glycosylation metabolic pathway; these interventions are directed towards reducing the immunogenicity for man of the glycoconjugates that are produced in heterologous systems. These modified cell lines can be used for the production of glycoconjugates for use in treatment in man.

The glycosylation of proteins affects a large number of their properties. Complex oligosaccharide structures are bound to specific residues of Asn (Asparagine) in a process known as N-glycosylation, which takes place between the endoplasmic reticulum (ER) and the Golgi vesicular system and is governed by a battery of enzymes such as glycosidase and glycosyl transferase. At the level of the individual glycoprotein, the oligosaccharide not only affects the biological activity, the folding, and the location and immunogenicity of the molecule to which it is bound, but also has a profound effect on the adhesion characteristics between the glycoconjugate and other proteins, macromolecular complexes, or cells. At systemic level, these saccharide structures take part in molecule and/or cell recognition phenomena during embryonic development, in viral infection processes, in inflammatory reactions, in hormonal action, in forms of arthritis, in the immune response, and in metastasis, to give only a few examples. Various examples of human genetic diseases associated with defects in the maturation of N-oligosaccharides also bear witness to the essential role of these glycan structures in animal development and physiology (9).

The sialic acids constitute a family of about 40 derivatives of the sugar with 9 carbon atoms which is known as neuraminic acid. The non-substituted form of neuraminic acid does not actually exist in nature. The amino group is generally acetylated to give N-acetylneuraminic acid (Neu5Ac):

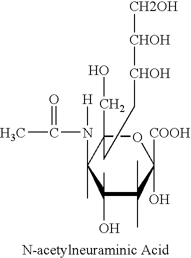

Neu5Ac represents the most common form of sialic acid. Replacing one of the hydrogen atoms in the methyl portion of the acetyl group with a hydroxyl group gives N-glycolylneuraminic acid:(Neu5Gc):

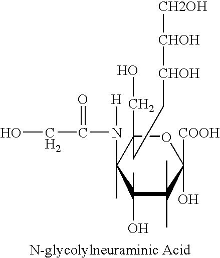

Neu5Ge is common in many animal tissues but has been found in the human species solely in certain forms of cancer.

The sialic acids are present practically throughout the animal kingdom, from the echinoderms to humans. They have an important role at structural level and perform important functions connected with cell and molecule recognition phenomena. By virtue of the terminal position and of the negative charge, they act as a protection for the subterminal portion of the molecule or of the cell and it is by virtue of the repulsion between their negative charges that the sialic acids can establish the correct conformation of the membrane proteins or enzymes of which they form part.

With regard to the recognition processes, the immune system can also distinguish self structures from non-self structures by virtue of their composition in terms of sialic acids. They are essential components of receptors for molecules such as hormones and cytokines; many pathogenic agents such as toxins, viruses, bacteria, and protozoa bind to and invade the host cell by means of receptors containing sialic acids.

Interest in the development of processes for the production of recombinant proteins which are of therapeutic interest in animal cells has given rise to considerable progress in the definition of highly efficient expression plasmids. Efforts directed towards optimizing the expression cell lines have been less significant. Currently, cells of the CHO (Chinese hamster ovary) line are considered optimal candidates as expression hosts since they are easily manipulable genetically, suitable for large-scale growth in suspension, and capable of producing glycoproteins the oligosaccharide portions of which are very similar, if not identical, to those found in man. Both the quantitative characteristics (incomplete glycosylation) and the qualitative characteristics (type of monosaccharides added to the oligosaccharide and type of bond) are important for the pharmacokinetic characteristics of glycoproteins for use as therapeutic agents. For example, it has been demonstrated that, in the case of glycoproteins, the pharmacokinetic properties of which are greatly dependent on the structure of the N-type saccharide portion, insufficient or incorrect sialylation or galactosylation may give rise to more rapid clearance of the proteins. In this connection, Weikert S. et al. (14) engineered two lines of CHO, one expressing stably the glycoprotein TNFR-IgG (fusion between Tissue Necrosis Factor Receptor and IgG1), and one expressing stably the glycoprotein TNK-tPA (a variant of the tissue Plasminogen Activator) for increasing the expression of two enzymes which are already normally present in those cells: β1,4-galactosyl transferase (GT) and α2,3-sialyl transferase (ST). They thus achieved more complete processing of the terminal portions of the oligosaccharides bound to the proteins TNFR-IgG and TNK-tPA with consequent slowing-down of the clearance.

At qualitative level, the glycosylation in CHO, in comparison with that found in human tissues, has two substantial differences relating to the sialylation:
1. in man, in addition to the α2,3-sialyl transferase (α2,3-ST) enzymatic activity which is also found in CHO cells, α2,6-sialyl transferase (α2,6-ST) enzymatic activity is present; as a result, whereas glycans of human origin have terminal sialic acids bound to the subterminal residue by means of both α2,3 and α2,6 bonds, in the glycans produced in CHO, the sialic acids are bound solely by α2,3-type bonds (2);
2. Neu5Gc is produced by the hydroxylation of Neu5Ac by the enzyme CMP-NeuAc hydroxylase (CMAH) (EC1.14.13.45).

In man, the gene encoding for this enzymatic activity is not functional. As a result NeuGc is absent in healthy human tissues, although traces of it have been found in some tumours. Glycoconjugates containing NeuGc are therefore able to induce an immune reaction in man and cause the formation of antibodies known as Hanganutziu-Deicher antibodies. CHO cells produce glycoproteins in which NeuGc represents about 3% of the total sialic acids (7) but the quantity of NeuGc present in the recombinant proteins may vary in dependence on factors such as the nature of the protein expressed, the site of insertion of the recombinant gene in the host's genome, and the cell cultivation conditions. The existence of clones of CHO in which NeuAc has been completely replaced by NeuGc is documented (8).

With regard to the presence of CMP-NeuAc hydroxylase enzymatic activity in CHO, it is to be considered undesirable from the point of view of the production of recombinant glycoproteins for use in therapy in man. Chenu S. et al. (3) have produced a CHO line which is free of the above-mentioned enzymatic activity. They used the CHO UH line of (2) above to produce a second stable cell line capable of producing antisense RNA directed against the messenger RNA of CMAH. CHO-UH cells were transfected with the use of a method derived from the in vitro amplification system (see above). In particular, the cells were transfected with concatenamers of DNA constituted by multiple copies of the expression box for the preselected antisense RNA and copies of the expression box for the enzyme dhfr in a ratio of about 10:1. The cell line HCO-AsUH2, in which the CMAH activity is reduced by 78% in comparison with the starting CHO-UH line, was thus obtained; this reduction in enzymatic activity corresponds to an equal reduction in the quantity of NeuGc in comparison with the parent line (NeuGc changes from 3.7±0.3% of the total sialic acids in CHO-UH to 0.62±0.05% in CHO-AsUH2). The reduction in CMAH activity in the new cell line is not total and is closely connected with the constant presence of antisense RNA within the cell, that is, with the continuity of the transcription of the sequence encoding for the antisense RNA.

### DESCRIPTION OF THE INVENTION

The object of the present invention is to provide a CHO cell line which does not produce Neu5Gc as a result of the hydroxylation of Neu5Ac.

The strategy followed by the inventors was to modify the CHO cell line genetically so as to deprive it of CMP-NeuAc hydroxylase activity and thus render it usable for the production of recombinant glycoconjugates of therapeutic interest usable in human therapy and having a lower immunogenic potential for man than the corresponding glycoconjugates produced in cells that have not been genetically modified.

This result has been achieved by eliminating from the genome of the CHO cell line a well-defined portion of the gene which encodes for CMAH, that is, the gene sequence which encodes for the catalytic domain of CMAH. In greater detail, the gene sequence which encodes for the sites of binding to the substrate (CMP-N-acetylneuraminic acid) and to the cofactor (cytochrome b5) has been eliminated from the genome of the CHO cell line.

The deletion of this sequence was performed by the method defined as “gene knock-out” or “gene targeting”, which is described, for example, in (4), (10), (11) and (13), which are incorporated herein by reference.

The deletion was thus brought about of a portion of the gene encoding for CMAH disposed between part of exon 10 and part of exon 15 (that is, eliminating exons 11, 12, 13 and 14 entirely) and, in greater detail, of the portion which corresponds to the sequence disposed between bases 787 and 1598 of the cDNA of CMAH and which corresponds to the portion of the gene encoding for CMAH the cDNA of which has the sequence given below (SEQ ID NO 1):

tcctgaaatggacacatgcattatcgtagagtacaaaggtcataaaatac
tcaacacagtggattgcaccagacccaatgggggaaggctgcctgagaaa
gctgctctaatgatgagtgattttgctggaggagcatcaggctttccaat
gactttcagcggtggaaaatttactgaggaatggaaggcccagttcatta
aggcggaaagaagaaagcttctgaattacaaagctcagctcgtgaaggac
ctccagcctcgaatctactgtccctttgctgggtattttgtggaatctca
tccatctgacaagtatattaaggaaacaaacatcaaaaatgacccgattc
aactcaacaatctcatcaagaaaaactgtgatgtggtgacatggacccca
cgacctggagctactcttgacctgggcaggatgctgaaggacccaacaga
cagccagggcatcatagagcctccagaagggacaaaaatttacaaggatt
catgggacttcggcccatacctgagcaccttgcactctgctgtaggagat
gaaatcttccttcactcgtcctggataaaagagtacttcacttgggctgg
atttaagagttacaacttggtggtcaggatgattgaaacagatgaagact
tcaacccttttcctggagggtatgactatctggtggactttctagatttg
tcttttccaaaagaaagaccaagcagggagcatccctatgaagaaatccg
tagccgtgtggatgtcgtcaggtacgtggtgaagcacggtctgctgtggg
atgacctgtaca

This corresponds to the portion of the gene encoding for the sequence of CMAH disposed between amino-acid 262 and amino-acid 532 and having the sequence given below (SEQ ID NO 2):

hpemdtcii veykghkiln tvdctrpngg rlpekaalmm
sdfaggasgf pmtfsggkft eewkaqfika errkllnyka
qlvkdlqpri ycpfagyfve shpsdkyike tnikndpiql
nnlikkncdv vtwtprpgat ldlgrmlkdp tdsqgiiepp
egtkiykdsw dfgpylstlh savgdeiflh sswikeyftw
agfksynlvv rmietdedfn pfpggydylv dfldlsfpke
rpsrehpyee irsrvdvvry vvkhgllwdd ly

The sequence of the cDNA of CMAH of Cricetulus griseus (the organism from which the CHO cell line was obtained) which was used for the purposes of the present invention is deposited in the NCBI data bank under accession number AJ242835.

As can be confirmed from the following pages, this method involved the replacement of a large part of the CMAH encoding sequence with exogenous DNA, for example, DNA which can confer resistance to specific antibiotics such as, for example zeocine; this approach, which extends to both alleles, led to complete and virtually irreversible silencing of the CMAH encoding gene.

Materials and Methods 1) Cloning of the 3′ Flanking Sequence for the Targeting of the CMAH Gene

Genome DNA extracted from CHO UH α2,6 ST⁺dhfr³¹ cells (2) was amplified with the following oligonucleotides:

| | |
|---|---|
| OL155 | |
| 5′ TTGGCTTCCAGACCCGGTTGCAGC 3′ | (Forward) |
| OL156 | |
| 5′ TTAGAGGGAGTTTTATCTGAAAATGATTCC 3′ | (Reverse) |

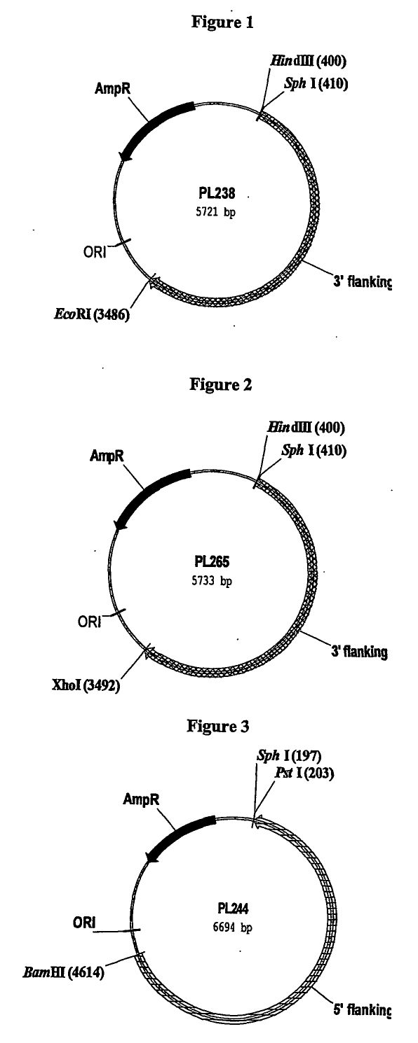

OL155 paired with a sequence belonging to exon 15 of the CMAH gene (in accordance with the exon numbering given for the human CMAH gene, see NCBI, UniGene Hs.24697) and OL156 paired with a region belonging to exon 16. The PCR fragment thus obtained (about 3.5 kb) was purified from gel and its ends were sequenced with the same oligonucleotides. The existence of an SphI site in the vicinity of the 5′ end of the fragment and of an EcoRI site in the vicinity of the 3′ end were thus shown. The uniqueness of the sites which was put in evidence by sequencing was proved by digestion of the fragment with the above-mentioned enzymes. The fragment thus digested was then cloned in the SphI and EcoRI sites of the vector pUC18 which carries the gene for resistance to ampicillin, NCBI Accession Number L08752 (15). After transformation of Escherichia coli strain DH5α (5), plasmid PL238 was obtained (FIG. 1). The cloned region was sequenced entirely.

PL238 was then modified by the insertion of a unique XhoI site in place of EcoRI. In particular, the synthetic oligonucleotide OL192, phosphorylated at 5′:

5′ Pho-AAT TGC TCG AGC 3′

was subjected to a cycle of heating to 95° for 5 minutes/slow cooling (about 3 hours) so as to obtain the following double-helix oligonucleotide, containing the XhoI site:

[symbol]

This oligonucleotide was cloned in the plasmid PL238 digested with EcoRI. After transformation of the DH5α strain, plasmid PL265 was obtained (FIG. 2). This plasmid differs from PL238 by the replacement of the single EcoRI site (downstream of the cloned 3′ flanking sequence) with a single XhoI site.

2) Cloning of the 5′ Flanking Sequence for the Targeting of the CMAH Gene

Genome DNA extracted from CHO UH α2,6 ST⁺dhfr⁻ cells (2) was amplified with the following oligonucleotides:

OL178 (Forward)
5′ [symbol] 3′
OL180 (Reverse)
5′ [symbol] 3′

OL178 paired with a sequence belonging to exon 8 of the CMAH gene and OL180 paired with a region belonging to exon 10. The PCR fragment thus obtained (about 4.4 kb) was digested with the enzymes BamHI and PstI (belonging to the oligonucleotides used for the amplification) and cloned in the same sites of PL42 (derived from pUC18 by removal of the Lac promoter therefrom). After transformation of E. coli strain DH5α, plasmid PL244 was obtained (FIG. 3).

PL244 was then modified for the insertion of two single RsrII and NotI sites.

In particular, the synthetic oligonucleotides OL193 and OL194, phosphorylated at 5′, were subjected to a cycle of heating to 95° C. for 5 minutes/slow cooling (about 3 hours) so as to obtain the following double-helix oligonucleotide, containing the RsrII site:

[symbol]

This oligonucleotide was cloned in plasmid PL244 digested with SphI. The above-mentioned synthetic oligonucleotide can be inserted in the SphI site in both orientations but only that shown is useful for the purposes of the invention. After transformation of the DH5α strain, the plasmid DNA was extracted from several ampicillin-resistant clones. By sequencing of the extracted DNAs, as expected, a frequency of 50% for each of the two orientations was found and a clone with the oligonucleotide inserted in the desired direction was selected. The plasmid thus obtained was digested with BamHI and ligated with the following double-helix synthetic oligonucleotide, phosphorylated at its 5′ ends, obtained as described above (heating to 95° C. and slow cooling):

[symbol]

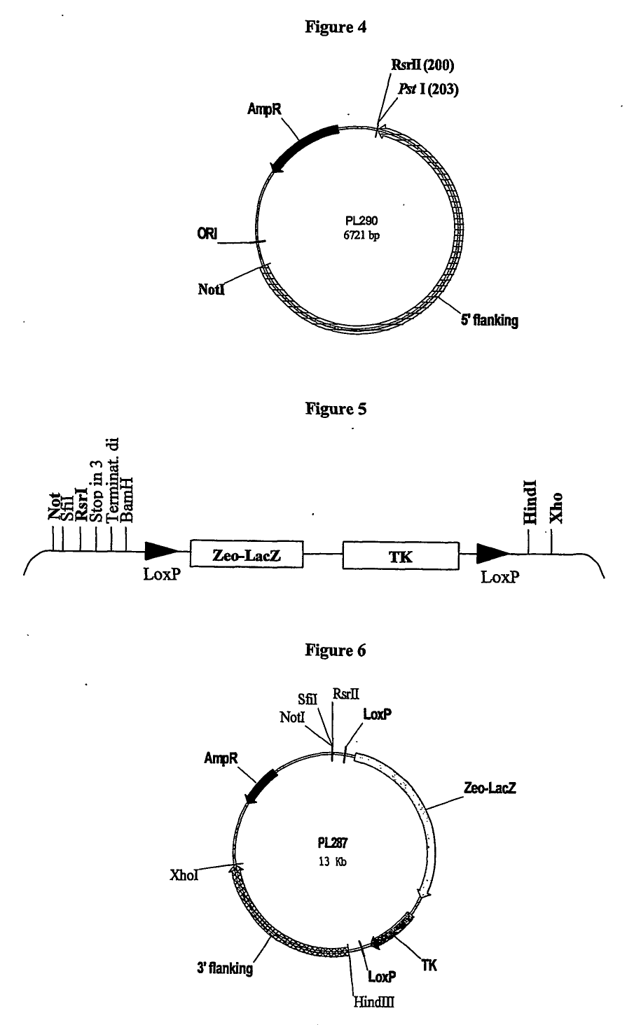

PL290 was thus obtained (FIG. 4).

3) Cloning of the 3′ Flanking and 5′ Flanking Regions in the Vector for the Targeting of the CMAH Gene

The plasmid PL286 in which the 3′ and 5′ flanking regions had been cloned had the structure given in FIG. 5 (the single sites to be used subsequently are shown).

This plasmid is composed of the following elements:
skeleton derived from a vector belonging to the commercial pBluescript plasmids family (Stratagene);
left polylinker in which a transcription terminator for eukaryotic cells and three STOP codons are also present in the three possible reading frames;
a Zeo-LacZ positive selection marker, NCBI Accession Number M81126 (1) which confers resistance to the antibiotic zeocine;
a TK (thymidine kinase) negative selection marker NCBI Accession Number V00467 (12). See also NCBI No. AF090451 for plasmid pKOTK of Lexicon Genetics INC. (1998) which confers sensitivity to gancyclovir;
a right polylinker;
two LoxP sites, NCBI No. M10289 (6) which can recombine with one another by virtue of the enzyme Cre recombinase.

The 3′ flanking region of the CMAH genome excised from the plasmid PL265 by digestion with the enzymes HindIII and XhoI, was cloned in the same sites of PL286. PL287 was thus obtained (FIG. 6).

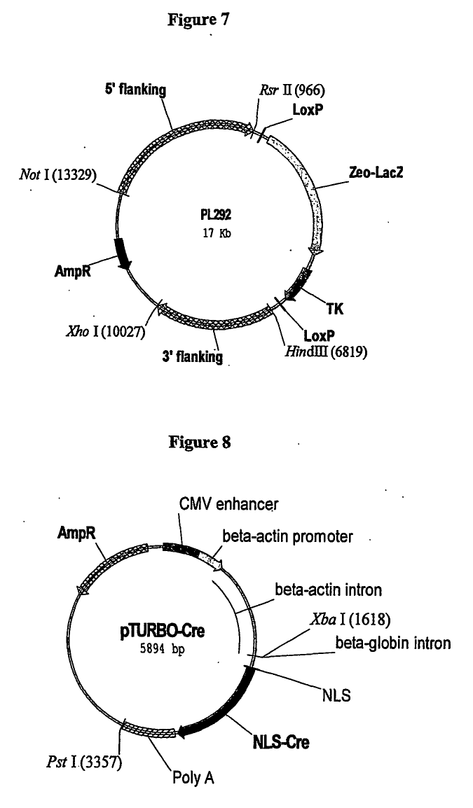

The 5′ flanking region of the CMAH gene, excised from plasmid PL290 by digestion with the enzymes NotI and RsrII, was cloned in the same sites of PL287. PL292 was thus obtained (FIG. 7).

4) Transfection of CHO α2,6-ST⁺ dhfr⁻ cells with PL292 and selection of stable clones

5×10⁵ CHO α2,6-ST⁺ dhfr⁻ cells (2) were seeded in wells of a multi-well plate with 6 wells (3.5 ml of culture for each well) in α-MEM medium (Gibco) supplemented with 10% foetal bovine serum, neomycin 0.45 mg/ml, penicillin 100 units/ml, and streptomycin 0.1 mg/ml. After incubation for 24 hours at 37° C. and 5% CO₂, the medium was replaced with fresh medium and transfection was carried out. The following mixtures were prepared:

| | | |
|---|---|---|
| Mix A: | NaCl 0.15 N | 155 μl |
| | PEI (polyethylene imine) | 45 μl |
| Mix B | NaCl 0.15 N | 190 μl |
| | circular PL292 | 10 μl (15 μg) |

Mix A was added to Mix B and, after incubation for 15 minutes, the final mixture (400 μl) was dispensed into the single well; the 6-well multi-well plate was then centrifuged for 5 minutes at 200×g to facilitate contact between the DNA-PEI complexes and the cell layer in the base of the well. After incubation for 48 hours, the cells were detached with Trypsin (about 0.75 mg for each well) and transferred to 90 mm Petri dishes in the presence of medium supplemented with zeocine (400 μg/ml final) for the selection of clones that were stably resistant to the above-mentioned antibiotic.

5) Screening to Identify Clones Which Have Undergone the Targeting of the CMAH Gene

The clones that were stably resistant to zeomycin were subjected to PCR screening. The genome DNA was extracted from pairs of clones and amplified with the following primers:

OL232: 5′CATGGACCTCAAGTTGGGAG 3′ (Forward)

This primer pairs on exon 8 upstream of the portion contained in the 51′ flanking region cloned in the plasmid PL292 used for the targeting.

RV Zeo: 5′GGAACGGCACTGGTCAACTT 3′ (Reverse)

This primer pairs in the initial portion of the Zeo-LacZ gene, which is a positive selection marker contained in plasmid PL292.

OL234: 5′TGAAAGTCATTGGAAAGCCTGATG 3′ (Reverse)

This primer pairs on exon 11 which should be missing in the allele of the CMAH gene which has undergone targeting.

The pair OL232+RV Zeo was used to provide evidence that the targeting event had taken place (amplification of the knock-out allele, appearance of a band of slightly less than 5000 bp); the pair OL232+OL234 was used as a check of the amplification capability of the genome DNA extracted (amplification of the natural allele, appearance of a band of slightly more than 5000 bp). Evidence was thus provided of a clone which had undergone the targeting of a single CMAH allele (a heterozygote clone).

6) Treatment of the Heterozygote Clone with Cre-Recombinase and Targeting of the Second CMAH Allele

The heterozygote clone was transfected transiently with the plasmid pTURBO-Cre, NCBI Accession Number: AF334827 (FIG. 8).

After a suitable incubation period, the cells thus treated were placed in the presence of gancyclovir. This molecule is converted, in the presence of the enzyme encoded by the gene TK, into a compound which is toxic to cells. The loss of the gene TK as a result of the recombination between the LoxP sites would allow the cells to survive. The genome DNA of the clones which were resistant to that antibiotic were amplified by PCR with primers capable of providing evidence that recombination had taken place between the LoxP sites. A clone in which that recombination had occurred was thus selected and was subjected to a second cycle of transfection with PL292 for the targeting of the second allele (see sections 4 and 5), followed by a transient transfection cycle with pTURBO-Cre to obtain a clone in which:
1) both of the alleles of the CMAH gene had undergone the targeting;
2) the sequences used for the positive selection (Zeo-LacZ) and the negative selection (TK) had been eliminated.

The strain obtained was then subjected to biochemical tests to quantify NeuAc and NeuGc in cell and/or recombinant glycoproteins.

### BIBLIOGRAPHIC REFERENCES

1. Baron M, Reynes J P, Stassi D and Tiraby G “A selectable bifunctional beta-galactosidase::phleomycin-resistance fusion protein as a potential marker for eukaryotic cells” Gene 114 (1992) 239-243.
2. Bragonzi A, Distefano G, Buckberry L D, Acerbis G, Foglieni C, Lamotte D, Campi G, Marc A, Soria M R, Jenkins N and Monaco L “A new Chinese hamster ovary cell line expressing alpha2,6-sialyl transferase used as universal host for the production of human-like sialylated recombinant glycoproteins” Biochim Biophys Acta 1474 (2000) 273-82.
3. Chenu S, Gregoire A, Malykh Y, Visvikis A, Monaco L, Shaw L, Schauer R, Marc A, and Goergen J L “Reduction of CMP-N-acetylneuraminic acid hydroxylase activity in engineered Chinese hamster ovary cells using an antisense-RNA strategy” Biochim. Biophys. Acta 1622 (2003) 133-144.
4. Galli-Taliadoros L A, Sedgwick J D, Wood S A and Korner H “Gene knock-out technology: a methodological overview for the interested novice” J. Immunol. Methods 181(1995) 1-15.
5. Hanahan D “Studies on transformation of Escherichia coli with plasmids” J. Mol. Biol. 166 (1983) 557-580.
6. Hoess R H, Ziese M and Sternberg N “P1 site specific recombination: nucleotide sequence of the recombining sites” Proc. Natl. Acad. Sci. U.S.A. 79 (1982) 3398-3402.
7. Hokke C H, Bergwerff A A, van Dedem G W, van Oostrum J, Kamerling J P and Vliegenthart J F “Sialylated carbohydrate chains of recombinant human glycoproteins expressed in Chinese hamster ovary cells contain traces of N-glycolylneuraminic acid” FEBS Lett. 275 (1990) 9-14.
8. Hubbard S C, Walls L, Ruley H E and Muchmore E A “Generation of Chinese Hamster Ovary Cells Glycosylation Mutants by Retroviral Insertional Mutagenesis” The Journal of Biol. Chem. 269 (1994) 3717-3724.
9. Moremen K W “Golgi alpha-mannosidase II deficiency in vertebrate systems: implications for asparagine-linked oligosaccharide processing in mammals” Biochim Biophys Acta. 1573 (2002) 225-235.
10. Smith C M “Gene-targeting strategies provide an avenue for studying gene function” The Scientist 14 (2000) 32-36.
11. Sternberg N, Sauer B, Hoess R and Abremski K “Bacteriophage P1 cre gene and its regulatory region. Evidence for multiple promoters and for regulation by DNA methylation” J. Mol. Biol. 20 (1986) 197-212.
12. Wagner M J, Sharp J A and Summers W C “Nucleotide sequence of the thymidine kinase gene of herpes simplex virus type 1” Proc. Natl. Acad. Sci. U.S.A. 78 (1981) 1441-1445.
13. Warner T G “Enhancing therapeutic glycoprotein production in Chinese hamster ovary cells by metabolic engineering endogenous gene control with antisense DNA and gene targeting” Glycobiology 9 (1999) 841-850.
14. Weikert S, Papac D, Briggs J, Cowfer D, Tom S, Gawlitzek M, Lofgren J, Mehta S, Chisholm V, Modi N, Eppler S, Carrol K, Chamow S, Peers D, Berman P and Krummen L “Engineering Chinese hamster ovary cells to maximize sialic acid content of recombinant glycoproteins” Nature Biotechnology 17 (1999) 1116-1121.
15. Yanisch-Perron C, Vieira J and Messing J “Improved M13 phage cloning vectors and host strains: nucleotide sequences of the M13mpl8 and pUC19 vectors” Gene 33 (1985) 1-119.

1-14. (canceled)

15. A CHO cell deprived of a portion of the gene encoding for CMAH.

16. A CHO cell according to claim 15 deprived of a gene sequence which encodes for the binding site to the substrate (CMP-N-acetyl-neuraminic acid) and for the binding site to the cofactor (b5 cytochrome).

17. A CHO cell according to claim 15 wherein said portion is disposed between part of exon 10 and part of exon 15 of the gene encoding for CMAH.

18. A CHO cell according to claim 15 wherein said portion is within encoding for the sequence disposed between bases 787 and 1598 of cDNA encoding for CMAH.

19. A cell according to claim 18, wherein said portion has the sequence: SEQ ID NO: 1.

20. A CHO cell according to claim 18 deprived of the portion of the gene encoding for the sequence of CMAH disposed between amino-acid 262 and amino-acid 532.

21. A CHO cell according to claim 18, wherein said portion has the sequence: SEQ ID NO: 2.

22. A CHO cell according to claim 18, wherein the NCBI accession number of the cDNA is AJ242835.

23. A CHO cell according to claim 15, wherein the portion of the gene encoding for CMAH is absent from both alleles.

24. A CHO cell according to claim 15, wherein the portion eliminated has been replaced by at least one DNA sequence encoding for resistance to an antibiotic.

25. A CHO cell according to claim 24, wherein the antibiotic is zeocine.

26. A method for expressing a heterologous recombinant protein comprising culturing CHO cells according to claim 15, said cells having been transformed to express said heterologous recombinant protein.

27. The method of claim 26, wherein said protein is at least one recombinant glycoconjugate.

28. A CHO cell deprived of the portion of the gene encoding the catalytic domain of CMAH.

29. A CHO cell according to claim 28 deprived of the gene sequence which encodes the binding site to the substrate (CMP-N-acetyl-neuraminic acid) and the binding site to the cofactor (b5 cytochrome).

30. A CHO cell according to claim 28 deprived of a portion of the gene encoding CMAH that is disposed between part of exon 10 and part of exon 15 and comprises exons 11 through 14 in their entirety.

31. A CHO cell according to claim 28 deprived of the portion of the gene encoding CMAH having the sequence disposed between bases 787 and 1598 of the cDNA and CMAH.

32. A CHO cell according to claim 31 deprived of a portion of the gene encoding CMAH, the cDNA of said portion having the sequence: SEQ ID NO: 1.

33. A CHO cell according to claim 31, said portion of the gene encoding the sequence of CMAH said portion disposed between amino-acid 262 and amino-acid 532.

34. A CHO cell acording to claim 31 said portion of the gene encoding the portion of CMAH having the sequence: SEQ ID NO: 2.

35. A CHO cell according to claim 31, wherein the NCBI accession number of the cDNA is AJ242835.

36. A CHO cell according to claim 28, wherein the portion CHO of the gene coding for CMAH is absent from both alleles.

37. A CHO cell according to claim 28, wherein the sequence eliminated has been replaced by at least one DNA sequence encoding for resistance to an antibiotic.

38. A CHO cell according to claim 37, wherein the antibiotic is zeocine.

39. A method for expressing a heterologous recombinant protein comprising culturing CHO cells according to claim 28, said cells having been transformed to express said heterologous recombinant protein.

40. The method of claim 39, wherein said protein is at-least one recombinant glycoconjugate.


SEQUENCE LISTING (2 sequences)

SEQ ID NO: 1 - DNA, 812 bp, Cricetulus griseus
tcctgaaatggacacatgcattatcgtagagtacaaaggtcataaaatactcaacacagtggattgcaccagacccaatgggggaaggctgcctgagaaagctgctctaatgatgagtgattttgctggaggagcatcaggctttccaatgactttcagcggtggaaaatttactgaggaatggaaggcccagttcattaaggcggaaagaagaaagcttctgaattacaaagctcagctcgtgaaggacctccagcctcgaatctactgtccctttgctgggtattttgtggaatctcatccatctgacaagtatattaaggaaacaaacatcaaaaatgacccgattcaactcaacaatctcatcaagaaaaactgtgatgtggtgacatggaccccacgacctggagctactcttgacctgggcaggatgctgaaggacccaacagacagccagggcatcatagagcctccagaagggacaaaaatttacaaggattcatgggacttcggcccatacctgagcaccttgcactctgctgtaggagatgaaatcttccttcactcgtcctggataaaagagtacttcacttgggctggatttaagagttacaacttggtggtcaggatgattgaaacagatgaagacttcaacccttttcctggagggtatgactatctggtggactttctagatttgtcttttccaaaagaaagaccaagcagggagcatccctatgaagaaatccgtagccgtgtggatgtcgtcaggtacgtggtgaagcacggtctgctgtgggatgacctgtaca

SEQ ID NO: 2 - protein, 271 aa, Cricetulus griseus
HPEMDTCIIVEYKGHKILNTVDCTRPNGGRLPEKAALMMSDFAGGASGFPMTFSGGKFTEEWKAQFIKAERRKLLNYKAQLVKDLQPRIYCPFAGYFVESHPSDKYIKETNIKNDPIQLNNLIKKNCDVVTWTPRPGATLDLGRMLKDPTDSQGIIEPPEGTKIYKDSWDFGPYLSTLHSAVGDEIFLHSSWIKEYFTWAGFKSYNLVVRMIETDEDFNPFPGGYDYLVDFLDLSFPKERPSREHPYEEIRSRVDVVRYVVKHGLLWDDLY